(19) United States
(12) Patent Application Publication
(10) Pub. No.: US 2005/0074156 A1
(43) Pub. Date: Apr. 7, 2005

(54) Automatic detection of radioactive seeds for CT based post-planning for prostate seed implantation based on the hough transform

(76) Inventors: Edward J. Holupka, Boston, MA (US); Everette C. Burdette, Champaign, IL (US)

Correspondence Address: THOMPSON COBURN, LLP, ONE US BANK PLAZA, SUITE 3500, ST LOUIS, MO, 63101, US

(21) Appl. No.: 10/769,059

(22) Filed: Jan. 30, 2004

Related U.S. Application Data
(60) Provisional application No. 60/444,105, filed on Jan. 30, 2003.

(51) Int. Cl. G06K 9/00; A61B 5/05

(52) U.S. Cl. 382/131; 600/427

(57) ABSTRACT

A technique for detecting implanted seeds automatically from CT image data. Various features of the inventive technique include (1) thresholding a 3D CT data set on a voxel basis; and (2) using the Hough transform to identify shapes within the thresholded data that match a predefined 3D shape corresponding to an implanted seed.

### CROSS REFERENCE AND PRIORITY CLAIM TO RELATED APPLICATION

This application claims the benefit under 35 U.S.C. §119(e) of provisional patent application Ser. No. 60/444,105 entitled “Automatic Detection of Radioactive Seeds for CT Based Post-Planning for Prostate Seed Implantation Based on the Hough Transform”, filed Jan. 30, 2003, the disclosure of which is incorporated by reference herein.

### FIELD OF THE INVENTION

The present invention is directed toward automated detection of seeds within CT images.

### BACKGROUND AND SUMMARY OF THE INVENTION

With the introduction of the Prostate Specific Antigen (PSA) test, many more patients are presenting with early stage prostate cancer. For these early stage diseases for which the cancer is totally isolated to the prostate gland one treatment option that has been gaining in popularity is radioactive seed implantation. Radioactive seed implants offer the possibility of excellent local control as well as minimal morbidity. Due to the excellent clinical results of seed implantation there has been much attention paid by the medical community toward improving the seed implantation process either by improving the specific technique itself or improving the cost effectiveness so that the procedure can be offered to a wider geographical range of patients.

One area where the seed implantation process can be improved is in the development of a fast and accurate CT or MR based post-plan dosimetry system. While it is true that once the seeds have been implanted, the determination of the quality of the implant after the fact can not alter the physical implant. However, much can be learned from a properly performed post-plan. The post-plan can indicate hastily dropped seeds resulting in inferior gland coverage or too much dose to the rectum or urethra. These quantitative results of the post-plan can be used by the physician to indicate that more care during the procedure must be taken. In addition, a properly performed post-plan that indicates inadequate dosimetric coverage of the gland indicates that a postoperative procedure may be considered to correct the situation. In some cases the patient can be reseeded to boost under dosed regions, or more radically, a salvage prostectomy may be performed.

There have been many algorithms developed over the past two years, which claim to automatically find seed centers on post operative CT studies. However, most of these algorithms require a significant amount of user intervention and expertise to achieve an adequate result. Additionally, most, if not all, of these algorithms begin with a simple thresholding of the CT data to reduce the CT images to binary images. Some algorithms end here. The thresholding results in groups of contiguous voxels which represent single or multiple seeds. If seeds are grouped together in physical space so that they are within the resolution of the CT scan, or more precisely, within the resolution of the artifacts created by each individual seed, then the resultant contiguous set of voxels will contain many more voxels than if the seeds were distinct within the CT scan. The difficult and most creative part of any automatic seed finder is in the reduction of these multiple seed voxel sets to individual seed center coordinates. In addition to determining the center of the seeds, determination of the seed direction is also of value. If an anisotropic dose calculation is to be performed, the seed center and direction must be determined.

Against this backdrop, the inventors herein have developed an improved seed detection algorithm. The seed detection algorithm comprises the steps of (1) thresholding a 3D CT data set on a voxel basis; and (2) using the Hough transform to identify shapes within the thresholded data that match a predefined 3D shape corresponding to an implanted seed. The 3D CT data set is reconstructed from the CT slice data. Further, the predefined shape used during the Hough transform process is preferably an ellipsoid with a major axis and a minor axis with dimensions corresponding to the implanted seed. The algorithm may implemented on an apparatus such as a programmed computer.

The algorithm of the present invention's use of the Hough transform with a 3D ellipsoidal template to determine seed centers and orientations represents a vast improvement in the seed detection art. The present invention's ability to determine seed orientation and split multiple seed centers is particularly advantageous. Further, the algorithm of the present invention is preferably fully automatic and determines the seed centers and directions as seen on post-operative CT scans of the prostate. The algorithm can be verified using a unique CT based phantom that incorporates an array of contiguous seeds so that they appear as one contiguous set of voxels on CT. In addition a quality assurance procedure is described to verify the accuracy of the seed finder both in phantom as well as in the clinical setting.

### BRIEF DESCRIPTION OF THE DRAWINGS

FIG. 1(a) illustrates an exemplary voxel representation of the Amersham 6711 ¹²⁵I seed;

FIG. 1(b) illustrates the ellipsoidal representation of the seed depicted in FIG. 1(a);

FIG. 2 is a parametric representation of a two-dimensional line;

FIG. 3(a) depicts a binary image containing seven lines;

FIG. 3(b) depicts the Hough transform of the binary image of FIG. 3(a);

FIG. 3(c) depicts the Hough transform of FIG. 3(b) with the contrast adjusted to display the underlying structure;

FIG. 4(a) illustrate a distribution of measured best-fit ellipsoidal parameters for the ellipsoid minor axis;

FIG. 4(b) illustrate a distribution of measured best-fit ellipsoidal parameters for the ellipsoid major axis;

FIG. 5 illustrates a multi-seed CT phantom;

FIGS. 6(a)-(d) illustrate DRRs and projections of the calculated seed positions;

### DETAILED DESCRIPTION OF THE PREFERRED EMBODIMENT

### METHODS AND MATERIALS

Three-Dimensional Images

A three dimensional image is specified by a collection of voxels and their corresponding pixel values where Iᵢⱼₖ is the pixel value of the voxel located at index i, j, k. Further, 0≦i≦Nₓ, 0≦j≦N_{y}, and 0≦k<N_{z}, where Nₓ, N_{y}, and N_{z} are the width, height, and length of the image in units of voxels. The pixel values are usually bounded such that 0≦Iᵢⱼₖ<2ⁿ where n is the number of bits per pixel. If there is a coordinate system associated with the image space then each voxel index (i,j,k) has an associated vector position, \vec{r}ᵢⱼₖ. To simplify notational text let I(\vec{r})=I(\vec{r}ᵢⱼₖ)=Iᵢⱼₖ. Let \vec{r}ᵢⱼₖ correspond to the spatial center of the voxel.

The algorithm presented here begins with the reduction of the CT image data to binary image data through a simple thresholding filter. The thresholding filter is given by

$$J ( \overline{r} ) = \{ \begin{matrix}0 & I ( \overline{r} ) < λ \\ 1 & I ( \overline{r} ) \geq λ\end{matrix} , where , 0 \leq λ \leq {2}^{n} - 1 , \qquad 1.0$$

where λ is the thresholding limit. Sets of super thresholded voxels (the voxels thresholded to “1” that are contiguous) will be referred to as “blobs”. Decreasing the value of λ will generally lead to more blobs as well blobs of greater size (i.e., number of voxels per blob). Preferably, λ is set near the white end of the grayscale, near the 80-90 percentile mark, depending upon the total bandwidth of the CT scanner.

There are now many different manufactures of radioactive seeds for prostate permanent seed implants. The possible isotope types include ¹²⁵I and ¹⁰³Pd. For the most part the seeds are all approximately the same in physical size and shape, that is small stainless steel cylinders of approximate dimensions 5 mm in length and 1 mm in diameter. However, they tend to have different characteristics under CT imaging. In the algorithm presented here, there are adequate parameters to model any commercially available seed.

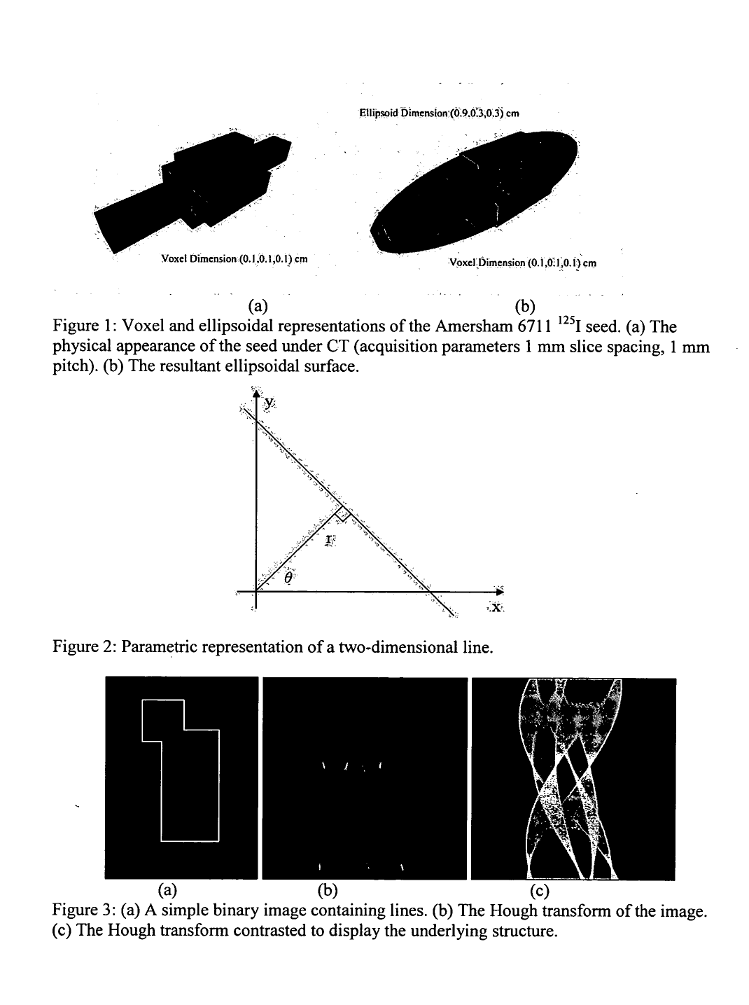

The seeds tend to create artifacts in the CT images due to their approximate singular physical nature. While the number and size of the subsequent blobs are strongly dependent on the CT acquisition parameters, assume for the subsequent analysis that the CT acquisition parameter results in a slice spacing of 1 mm . Slice spacing should be in the range of 1-5 mm, with the range 2-3 mm being preferable. After the thresholding filter described in equation 1 is applied, single seeds are represented by blobs of approximately 15 to 25 voxels in size. These single seed blobs can be fit to an ellipsoidal surface. The ellipsoid is a regular ellipsoid whose major axis is approximately 1.0 cm and minor axis is approximately 0.3 cm. The blobs that correspond to the actual seeds are larger on the CT images. This is due to the CT artifact associated with each seed. This artifact depends on the direction the longitudinal axis of the seed makes with respect to the scan direction as well as other CT acquisition parameters. FIG. 1(a) displays the resulting multivoxel blob for a single seed while FIG. 1(b) displays the fit ellipsoidal surface for the same multivoxel blob. If two or more seeds are within 1 mm of each other the resultant blob will be larger and in fact usually multidirectional. These proximal seeds will be represented by a large, single blob. It is exactly this situation that tests the true effectiveness and robustness of any automatic seed finder. While there exist academic and commercial automatic seed finders, they either tend to require much user intervention or do not accurately break these multi-seed blobs into their component seeds. In fact the authors have found no evidence in the literature of studies which attempt to quantitatively determine the accuracy of the breaking of multi-seed blobs or for that matter, no quality assurance of post-algorithm seed breaking in a clinical setting or in phantom.

Once the image has been thresholded, the resultant binary image is analyzed to determine the seed centers and directions. In order to achieve this a specialized Hough transform is applied.

The Hough Transform

The Hough transform was first applied to determine lines in an image. It is instructive to describe the line finding procedure for the purpose of clearly illustrating the concept of the Hough transform. Consider a two dimensional image containing a number of lines. A two dimensional line may be represented in a number of ways. For the description of the Hough transform considered here, the representation of the line will be given in terms of its angle, Ø, and the perpendicular distance, r. FIG. 2 displays the relationship of r and Ø to the line. FIG. 3(a) displays a binary image that contains a number of line segments. A line in an image is mathematically described as the collection of voxel indices (i,j) such that

$$j = tan θ \cdot i + \frac{r}{sin θ} . \qquad 1.1$$

The first step in the Hough transform is to collect all binary voxels indices whose value is one, that is the set S such that,
S={(i,j):∀vᵢⱼ=1}. 1.2
Now consider all possible pairs of such voxel indices {(i₁,j₁),(i₂,j₂)}. From this pair it is possible to calculate a perpendicular distance r and angle Ø which describes the line which passes through them. The parameter Ø and r are given by,

$$tan θ = \frac{{j}_{2} - {j}_{1}}{{i}_{2} - {i}_{1}} , and , r = {j}_{1} \cdot cos θ - {i}_{1} \cdot sin θ . \qquad 1.3$$

The Hough transform of the image is simply the set of all possible Ø and r values determined from the original binary image. FIG. 3(b) displays the Hough transform of the image displayed in FIG. 3(a). The axes of the Hough transform are the parameters Ø and r. The Hough transform contains a number of bright spots. These bright pixels correspond to the true Ø and r values of the lines in the original image. This is because the true lines in the image will contribute the most pixel pairs to the Hough Transformed Image. FIG. 3(c) displays the Hough transform of the image displayed in FIG. 3(a) except the contrast has been adjusted so as to display the underlying structure.

In the final step in determining the actual line parameters in the original image, the Hough transform must be thresholded to select only the bright pixels. Thresholding of FIG. 3(b) results in the determination of the true line parameters of image in FIG. 2(a).

The Ellipsoidal Hough Transform

The Hough transform is a generalized algorithm which can be applied to any three-dimensional object which can quantitatively be described. The ellipsoid is such an object. A general ellipsoid can be described as,

$$\frac{{( x - {x}_{0} )}^{2}}{{a}^{2}} + \frac{{( y - {y}_{0} )}^{2}}{{b}^{2}} + \frac{{( z - {z}_{0} )}^{2}}{{c}^{2}} = 1 , \qquad 1.4$$

where,
\vec{r}₀=(x₀,y₀,z₀), 1.5
describes the center of the ellipsoid and,
\vec{ζ}=(a,b,c), 1.6
describes the length and direction of the major and minor axes. As with the two-dimensional line finding algorithm, the algorithm which determines the ellipsoids contained within an image begins with a thresholding of the three-dimensional image. The thresholding parameter, λ, can be adjusted to improve the accuracy of the seed finder. Once the image is thresholded, all superthresholded voxels are collected into contiguous groups, or blobs. The traditional Hough transform operates on the entire image, whereas in the algorithm presented here, the Hough transform is applied to the subimage of each blob. This dramatically reduces the computation time required for the Hough transform. After all blobs have been collected, all possible triplets of voxels are generated for each blob. This results in a set of voxels indices represented by the set S_{E} given by,
S_{E}={(i₁,j₁,k₁),(i₂, j₂,k₂),(i₃,j₃,k₃):∀vᵢⱼₖ ∈blob}. 1.7
Assume that the point (i₁, j₁, k₁) represents a surface voxel on the ellipsoid and that the other two points, (i₂, j₂, k₂) and (i₃, j₃, k₃) represent the two foci of the ellipsoid, then if they are indeed surface and foci points their distances must satisfy the condition,
d₁₂+d₁₃+d₂₃=2·a, 1.8
where d₁₂ is the distance between point one and two, d₁₃ the distance between one and three, and d₂₃ the distance between two and three. If equation 1.8 is true for the triplet then they are part of an ellipsoid whose axes are (a,b,c). Otherwise the triplet is rejected. The criterion for acceptance is dependent on the voxel size. That is the equation of invariance must be satisfied with in the uncertainly of the size of the voxel. If they do satisfy the equation of geometric invariance, equation 1.8, then the center and direction of the ellipsoid are recorded and the next triplet is inspected for invariance. The center is given by,

$${\vec{r}}_{center} = \frac{1}{2} ( {\vec{r}}_{2} + {\vec{r}}_{3} ) , \qquad 1.9$$

and the direction relative to \vec{r}₂ is given by,

$${\vec{r}}_{direction} = \frac{{\vec{r}}_{2} - {\vec{r}}_{3}}{\vert {\vec{r}}_{2} - {\vec{r}}_{3} \vert} . \qquad 1.10$$

It is assumed that the values for a, b, and c are known a priori so that the search for geometric invariance can be performed.
Determination of the Ellipsoidal Axes Dimensions

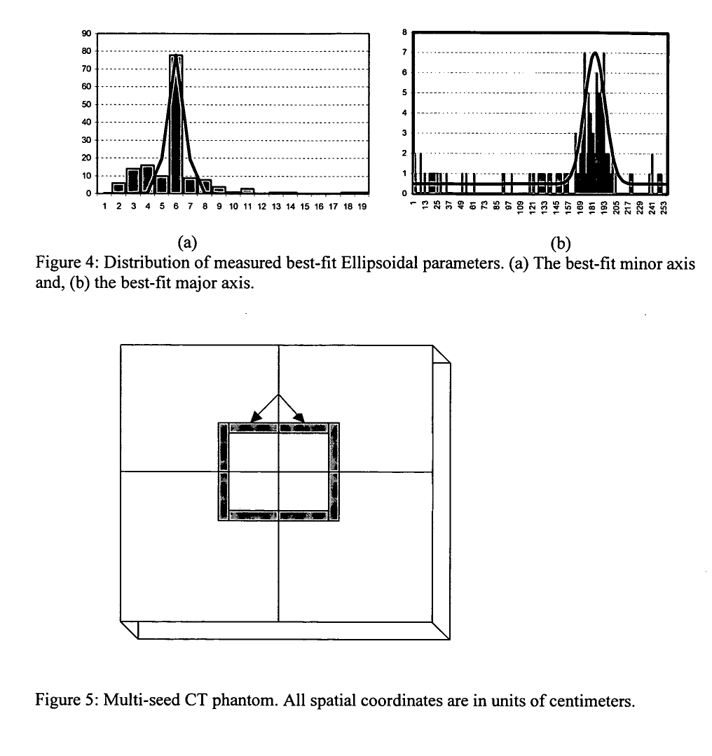

To determine the values of a, b, and c for Amersham Nycomed's ¹²⁵I model 6711, 100 patients who were treated with interstitial seed implantation using this model seed were analyzed. The post-operative CT scans were thresholded and the contiguous blobs collected. Each blob was then fit to an ellipse of the form given in equation 1.4 where b=c. The results of the best fit values were histogramed and appear in FIG. 4(a). Seeds which were too close and coalesced into a single blob were included in the analysis and would not represent the true values of the ellipsoid dimensions. However, this coalescence is the exception rather than the rule and would represent weak, distance peaks in the resultant histogram. Ignoring these exceptions, the average value for the length of the major and minor axes of the best fit ellipsoid for the model 6711 seed are a=1.0 and b=0.35. The best fit value for the axes show a minor dependence on the threshold parameter λ. FIG. 4(b) displays the best fit value for the axes as a function of λ, again for the model 6711 seed. The same techniques can be used to determine the a, b, and c parameters for other types of seed implants.

Phantom Test

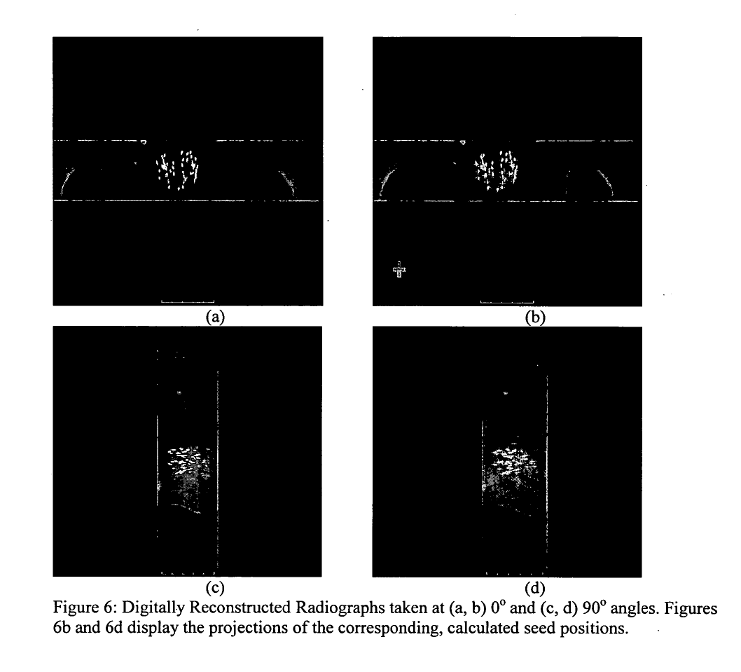

A phantom consisting of eight dummy (zero activity) 6711 seeds was constructed. FIG. 5 displays the phantom geometry along with the seed coordinates relative to the geometric center of the seeds. The phantom was constructed such that the seeds were in physical contact such that they described a square shape. The seeds were embedded in between to one inch thick sheets of superflab material. The seed phantom was scanned at 1 mm slice spacing throughout the entire phantom for a total of 64 CT slices. The resultant CT image set for this phantom displayed the seeds as one contiguous blob which contains the eight individual seeds. The automatic seed finding algorithm presented here was applied to the CT images of the phantom. A threshold value of λ=210 was applied to create the binary image and resultant, single, multi-voxel blob which contained 823 voxels.

Quality Assurance Digitally Reconstructed Radiograph (DRR)

In order to attempt to quality assure the results of the automatic seed finder a quality assurance tool based on a digitally reconstructed radiograph was developed by the authors. The tool is based on the construction of a DRR for any orientation and direction. The tool is very robust in this regard. It is not limited to the simple three orthogonal directions. The user simply chooses an initial direction for the DRR, usually the anterior to posterior (AP) direction. On this DRR the bony anatomy and the seeds can be easily seen. FIG. 6(a) displays the results of an AP DRR for an actual patient study. The software then projects the calculated position of the seeds in this DRR projection as colored crosses. FIG. 6(b) displays the DRR along with the project calculated seed positions. The display of the calculated seed positions can be toggled on and off so that the user can inspect whether or not a high density region appears under the colored cross. It is in this manner that the user can get an estimate of whether the calculated seed position is correct or not. If a cross is draw with no corresponding, underlying high density region, then the calculated position of that particular seed is in question. If there exists an ambiguity in the DRR such as one seed lying behind another seed and along the ray from the DDR source to the DRR projection plane then the user simply calculates another DRR and a different angle. These processes of multiple DRR reconstructions can be repeated until the user is confident in the results of the automatic seed finder are correct or incorrect.

Patient Results

The commercial algorithm has been used for 850 patients at this and associated clinics. The software has never failed to properly identify the number and subsequent position of all implanted seeds for all of these patients. The accuracy in the number of implanted seeds was simply evaluated by comparing the actual number of seeds implanted to that number which the algorithm found. The accuracy of the determined spatial seed coordinates were more difficult to quantitatively assess, however, each patient was reviewed using the qualitative DRR QA tool previously described. Using this tool it was determined that the algorithm corrected determined the position of each seed for all 850 patients.

While the present invention has been described above in relation to its preferred embodiment, various modifications may be made thereto that still fall within the invention's scope, as would be recognized by those of ordinary skill in the art. Such modifications to the invention will be recognizable upon review of the teachings herein. Accordingly, the full scope of the present invention is to be defined solely by the appended claims and their legal equivalents.

1. A method of detecting implanted seeds automatically from CT image data, the method comprising:
thresholding a 3D CT data set on a voxel basis; and
using the Hough transform to identify shapes within the thresholded data that match a predefined 3D shape corresponding to an implanted seed.

2. The method of claim 1 further comprising:
reconstructing the 3D CT data set from CT slice data.

3. The method of claim 2 wherein the predefined shape is an ellipsoid with a major axis and a minor axis with dimensions corresponding to the implanted seed.

4. An apparatus for detecting implanted seeds automatically from CT image data, the apparatus comprising:
a processor configured to (1) threshold a 3D CT data set on a voxel basis; and (2) use the Hough transform to identify shapes within the thresholded data that match a predefined 3D shape corresponding to an implanted seed.

5. The apparatus of claim 4 wherein the processor is further configured to reconstruct the 3D CT data set from CT slice data.

6. The apparatus of claim 5 wherein the predefined shape is an ellipsoid with a major axis and a minor axis with dimensions corresponding to the implanted seed.

7. A computer readable medium for detecting implanted seeds automatically from CT image data, the computer-readable medium comprising:
a code segment executable by a processor for thresholding a 3D CT data set on a voxel basis; and
a code segment executable by a processor for using the Hough transform to identify shapes within the thresholded data that match a predefined 3D shape corresponding to an implanted seed.

8. The computer-readable medium of claim 7 further comprising a code segment executable by a processor for reconstructing the 3D CT data set from CT slice data.

9. The computer-readable medium of claim 7 wherein the predefined shape is an ellipsoid with a major axis and a minor axis with dimensions corresponding to the implanted seed.
